(19) United States
(12) Patent Application Publication
(10) Pub. No.: US 2013/0251810 A1
(43) Pub. Date: Sep. 26, 2013

(54) PROCESS FOR PREPARING ORALLY ADMINISTERED DABIGATRAN FORMULATIONS

(71) Applicants: Sabine LANDERER, Rissegg (DE); Thomas FRIEDL, Ochsenhausen (DE)

(72) Inventors: Sabine LANDERER, Rissegg (DE); Thomas FRIEDL, Ochsenhausen (DE)

(73) Assignee: BOEHRINGER INGELHEIM INTERNATIONAL GMBH, Ingelheim am Rhein (DE)

(21) Appl. No.: 13/897,949

(22) Filed: May 20, 2013

Related U.S. Application Data
(63) Continuation of application No. 12/934,727, filed on Nov. 2, 2010, now abandoned.

(30) Foreign Application Priority Data
Mar. 28, 2008 (EP) 08153667.4

(51) Int. Cl. A61K 9/50; A61K 31/4439

(52) U.S. Cl. CPC A61K 9/5089; A61K 31/4439
USPC 424/490; 514/338; 427/2.14

(57) ABSTRACT

The invention relates to an improved process for preparing a new medicament formulation of the active substance dabigatran etexilate of formula I

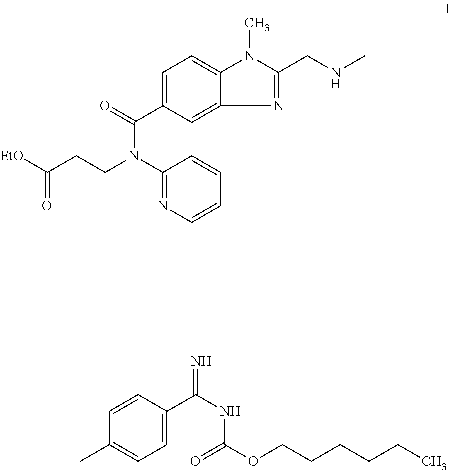

in the form of the methanesulphonic acid salt thereof, and this new medicament formulation as such.

The invention relates to an improved process for preparing a new medicament formulation of the active substance dabigatran etexilate of formula I

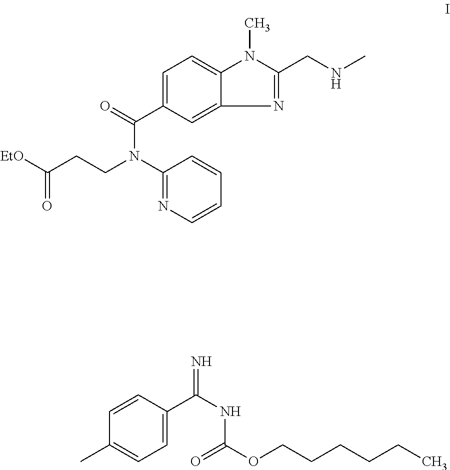

optionally in the form of the pharmaceutically acceptable salts thereof, as well as the new medicament formulation as such.

### BACKGROUND TO THE INVENTION

The compound of formula 1 is known from the prior art and was first disclosed in WO98/37075. It is a potent thrombin inhibitor which can be used for example for the post-operative prevention of deep vein thromboses and in stroke prevention, particularly for preventing strokes in patients with atrial fibrillation. WO 03/074056 discloses the methanesulphonic acid addition salt of dabiagtran-etexilate (ie: dabigatran etexilate methansulphonate) to be particularly useful.

The compound is usually administered orally. In particular, so-called pellet formulations may be used, as disclosed for example in WO 03/074056. These formulations are compositions, in which an active substance layer containing binder and optionally separating agent and surrounding a core material is applied to the substantially spherical core material, which consists of or contains a pharmaceutically acceptable organic acid. The core layer and the active substance layer are separated from one another by a so-called isolating layer. The schematic structure of an active substance formulation of this kind is shown in FIG. 1 of WO 03/074056.

The present invention relates to a process that can be used on an industrial scale for preparing active substance pellets containing dabigatran, which allows the formulation to be manufactured on a large scale. A further aim of the invention is to provide a process which allows the formulation to be manufactured with a reproducible quality.

According to WO 05/028468 the methansulphonic acid addition salt of dabiagtran etexilate exists in different polymorphic forms. It is another aim of the invention to provide for a manufacturing process which allows the manufacture of a pharmaceutical formulation that contains only one polymorphic form of the active ingredient dabigatran etexilat methansulphonate.

### DETAILED DESCRIPTION OF THE INVENTION

According to WO 05/028468 the methansulphonic acid addition salt of dabiagtran etexilate exists in different polymorphic forms. Surprisingly it has been found that polymorph I of dabigatran etexilate methanesulphonate is advantageous over polymorph II in view of its crystallisation properties. This allows polymorph I to be easier isolated and handled in and after the manufacturing process of the active ingredient. According to the invention, polymorph I is therefore the preferred polymorph.

In principle, different polymorphic forms of a substance may be characterized by different properties (including but not limited to stability, efficacy, processing properties during manufacture etc). As a matter of principle it is, therefore, advisable to produce a pharmaceutical composition that contains essentially only one polymorph.

Consequently, the invention relates to manufacturing process which allows the manufacture of a pharmaceutical formulation that contains essentially polymorph I of the active ingredient dabigatran etexilat methansulphonate.

The process according to the invention is characterised by a series of partial steps. First, the core 1 is produced from pharmaceutically acceptable organic acid. Within the scope of the present invention tartaric acid is used to prepare the core 1. The core material 1 thus obtained is then converted into so-called isolated tartaric acid cores 3 by spraying on an isolating suspension 2. A dabigatran suspension 4 prepared subsequently is sprayed onto these coated cores 3 in one or more process steps by means of a coating process. Finally, the active substance pellets 5 thus obtained are 1 packed into suitable capsules.

The isolated tartaric acid cores 3 should have a uniform, quasi-spherical geometry. Moreover they should have only minor potential defects in the isolation caused by satellites. The so-called satellites are small particles adhering to the outside of the otherwise rounded pellets and detracting from the otherwise quasi-spherical geometry of the pellets. The ideally spherical shape and low surface roughness is of particular importance for acid-sensitive active substances such as for example dabigatran etexilate, in which defects in the isolation caused by satellites that have broken off or by the excessively rough surface of over-large particles of tartaric acid powder may lead to significantly impaired storage stability and hence durability of the finished product. For this reason, with acid-sensitive active substances it is also essential to apply the isolating layer as such with high reproducibility and consistently high quality.

The core 1 is prepared from tartaric acid particles with a particle size in the range from 0.2-0.8 mm, preferably 0.3-0.7 mm, particularly preferably 0.4-0 6 mm (determined by air jet screening) onto which a solution of tartaric acid and binder is sprayed. The following method is used to prepare the solution. Tartaric acid is first of all dissolved in water together with a suitable binder, preferably with acacia (gum arabic) at elevated temperature, preferably at a temperature in the range from 30-70° C., particularly preferably in the range from 40-60° C. Preferably, 0.1-0.3 kg, particularly preferably 0.15-0.25 kg, particularly about 0.2 kg acacia are used per kilogram of tartaric acid put in. The amount of water is preferably 0.6-1.0 kg, preferably 0.7-0.9 kg, particularly about 0.8 kg per kilogram of tartaric acid put in.

Preferably, according to the invention, first of all a clear solution of acacia in water is prepared at the above-mentioned temperature. Once this has been obtained, the tartaric acid is then added preferably at constant temperature and while stirring continues. After the addition has ended the mixture is stirred for at least 1 hour, preferably between 3 and 10, particularly preferably 4-8, particularly preferably 5-6 hours.

The solution thus obtained is sprayed onto tartaric acid particles with a particle size of 0.2-0.8 mm, preferably 0.3-0.7 mm, particularly preferably 0.4-0.6 mm The proportion of particles with the above-mentioned particle size should be at least 90%, preferably at least 95%, particularly preferably at least 97%. For this, the tartaric acid particles are placed in a suitable container. The container is preferably a pan in which the particles are mixed and moved about by the rotation of the pan. Various designs of pan are known in the art and may optionally also be referred to as drum coaters. On this subject reference is made for example to the disclosures of EP 80199, WO 83/03052, WO 95/19713 or WO 06/134133. Within the scope of the present invention pans that may be used in the process according to the invention are optionally also known as horizontal pans.

The acid gum solution prepared by the method described hereinbefore is then sprayed onto the particles kept moving by rotation.

Within the scope of the present invention the material supplied for spraying is optionally also referred to as the pellet bed. The term pellet is to be regarded as equivalent to the term particle or core within the scope of the present invention.

According to the invention, preferably 0.8-1.6 kg, particularly preferably 1.0-1.4 kg, particularly preferably 1.2 kg of the above-mentioned acid gum solution are sprayed on per kilogram of tartaric acid particles supplied.

The amount of supply air in the process according to the invention is dependent on the batch size. The standardised amount of supply air per kilogram of tartaric acid cores supplied according to the invention is preferably in the range from 0.5-2 (m³/h)/kg, preferably 0.75-1.5 (m³/h)/kg, particularly preferably 0.9-1.1 (m³/h)/kg. By the amount of supply air is meant the amount of dry air introduced into the rotating pellet bed per hour. If for example 1000 kg tartaric acid cores are placed in one batch, a standardised amount of supply air of 1.0 (m³/h)/kg corresponds to an actual amount of supply air of 1000 m³/h. The temperature of the supply air fed in for drying according to the invention is preferably below 90° C., particularly preferably below 80° C. Ideally the temperature of the supply air should be in the range from 35°-75° C.

The pellet temperature (the temperature of the pellet bed formed) according to the invention is preferably in the range from 30-50° C., particularly preferably 36-44° C., ideally 38-42° C.

The differential pressure is preferably 1-3 mbar, particularly preferably 1.5-2.5 mbar, particularly preferably 1.8-2.2 mbar. The differential pressure is the pressure difference between the pan pressure and ambient pressure. The pan should preferably be at reduced pressure so that no acid dust escapes.

Spraying is carried out at a defined spray rate. By the spray rate is meant the amount of acid gum solution that is sprayed onto the rotating pellet bed per hour. The spray rate is dependant on the batch size in the process according to the invention. The standardised spray rate according to the invention per kilogram of tartaric acid crystals supplied is preferably in the range from 0.2-0.4 (kg/h)/kg, preferably 0.25-0.35 (kg/h)/kg, particularly preferably 0.28-0.32 (kg/h)/kg. If for example 1000 kg tartaric acid crystals are placed in one batch, a standardised spray rate of 0.3 (kg/h)/kg corresponds to an actual spray rate of 300 kg/h.

After a first portion of the acid gum solution has been sprayed onto the tartaric acid particles of particle size 0.2-0 8 mm and the solution has been distributed by rotating the pan, fine tartaric acid powder is sprinkled onto the moist tartaric acid particles. This tartaric acid powder consists of fine tartaric acid particles with a particle size of <100, preferably <75, particularly preferably <50 microns (determined by air jet screening). The proportion of particles with the above-mentioned particle size should be at least 85%, preferably at least 90%, particularly preferably at least 94%. According to the invention preferably 0.4-1.2 kg, particularly preferably 0.6-1.0 kg, particularly preferably 0.8 kg of the above-mentioned tartaric acid powder are used per kilogram of tartaric acid particles supplied. After sprinkling with the above-mentioned tartaric acid powder the material for spraying is dried until a product temperature of about 30-50° C., preferably about 40° C. is reached. After this, the acid gum solution is sprayed on again.

To ensure the uniform formation of spherical particles, the spraying on of the acid gum solution and the sprinkling with tartaric acid powder are carried out alternately. The total amounts of acid gum solution and tartaric acid powder are supplied in at least 100, preferably 150 to 350, particularly preferably 200 to 300, particularly preferably about 250 batches of similar size and the process steps described hereinbefore are repeated a corresponding number of times.

Once the process has ended, the cores 1 obtained are dried. The drying is preferably carried out at a temperature of 50-70° C., preferably 55-65° C. over a period of 24-72 hours, preferably 36-60 hours.

After the preparation of the tartaric acid cores 1 so-called isolation of the core material is necessary. An isolating layer is applied around the tartaric acid core, preventing any interaction of active substance with tartaric acid core in the later product.

The core material is isolated by spraying an isolating suspension 2 onto the tartaric acid cores 1 obtained by the process described hereinbefore. To prepare the isolating suspension 2 ethanol is placed in the batch container and hydroxypropylmethylcellulose and dimethylpolysiloxane are added and dissolved therein with stirring, then talc is added and suspended.

The use of hydroxypropylmethylcellulose and talc has proved superior to the use of gum arabic and talc, for example. By using hydroxypropylmethylcellulose together with talc it is possible to produce an isolating layer of constant quality in a reproducible manner. This quality and reproducibility has been tested on an industrial scale.

To prepare the isolating suspension 2, preferably 0.04-0.06 kg, particularly preferably 0.046-0.05 kg hydroxypropylmethylcellulose are used per kilogram of ethanol. Besides the use of hydroxypropylmethylcellulose it has proved particularly preferable according to the invention to add dimethylpolysiloxane to the isolating suspension 2 to prevent foaming. The amount of dimethylpolysiloxane which is added with stirring to the preparation of the isolating suspension 2 is preferably 0.6-1.2 g, particularly preferably 0.8-0.9 g per kilogram of ethanol. Finally talc is added and suspended therein with stirring. Preferably 0.04-0.06 kg, particularly preferably 0.046-0.05 kg talc are used per kilogram of ethanol.

In one aspect the present invention relates to an ethanolic isolating suspension 2 which contains hydroxypropylmethylcellulose, preferably in the above-mentioned quantities. In another aspect the present invention relates to an ethanolic isolating suspension 2 which, besides hydroxypropylmethylcellulose, contains dimethylpolysiloxane, preferably in the above-mentioned quantities. In another aspect the present invention relates to an ethanolic isolating suspension 2 which, besides hydroxypropylmethylcellulose and dimethylpolysiloxane, also contains talc, preferably in the above-mentioned quantities. In another aspect the present invention relates to an ethanolic isolating suspension 2 which may be obtained by the method described hereinbefore.

In another aspect the present invention relates to the use of the ethanolic isolating suspension 2, for isolating tartaric acid cores 1. In another aspect the present invention relates to the use of the ethanolic isolating suspension 2 as a starting material for preparing a medicament formulation of dabigatran etexilate methanesulphonate.

The isolating suspension 2 thus prepared is sprayed onto the previously prepared tartaric acid pellets 1 in a continuous spray process in a conventional horizontal coater. 0.5-0.8 kg, preferably 0.55-0.75 kg, particularly preferably 0.6-0.7 kg of isolating suspension are sprayed on per kilogram of tartaric acid cores 1 supplied.

The spraying is carried out at a defined spray rate. By the spray rate is meant the amount of isolating suspension 2 sprayed onto the pellets 1 per hour. The spray rate in the process according to the invention is dependent on the batch size. The standardised spray rate according to the invention is preferably in the range from 0.01-0.1 (kg/h)/kg, preferably 0.02-0.04 (kg/h)/kg, particularly preferably 0.025-0.035 (kg/h)/kg per kilogram of tartaric acid pellets 1 supplied. If for example 1200 kg tartaric acid cores are placed in one batch, a standardised spray rate of 0.027 (kg/h)/kg corresponds to an actual spray rate of 32 kg/h. If for example 600 kg tartaric acid cores are placed in one batch, a standardised spray rate of 0.035 (kg/h)/kg corresponds to an actual spray rate of 21 kg/h.

During this continuous process the cores are dried continuously with a supply of air at up to 70° C., preferably from 25-70° C.

By the amount of supply air is meant the amount of dry air that is introduced into the rotating pellet bed per hour. The amount of supply air in the process according to the invention is dependant on the batch size. The standardised amount of supply air according to the invention is preferably in the range from 1.0-2.5 (m³/h)/kg. Preferably 1.2-2.0 (m³/h)/kg, particularly preferably 1.40-1.85 (m³/h)/kg per kilogram of tartaric acid cores 2 originally supplied. If for example 600 kg tartaric acid cores 2 are placed in one batch, a standardised amount of supply air of 1.83 (m³/h)/kg corresponds to an actual amount of supply air of 1100 m³/h. If for example 1200 kg tartaric acid cores 3 are placed in one batch, a standardised amount of supply air of 1.42 (m³/h)/kg corresponds to an actual amount of supply air of 1700 m³/h.

In another aspect the present invention relates to the isolated tartaric acid cores 3 as such which are obtained by the above process.

The isolated tartaric acid cores 3 which may be obtained according to the invention have a uniform, quasi-spherical geometry which makes further processing considerably easier. Furthermore, the pellets 3 according to the invention have only minor potential defects in the isolation caused by so-called satellites. The so-called satellites are small particles adhering to the outside of the otherwise rounded pellets and detracting from the otherwise quasi-spherical geometry of the pellets. The ideally spherical shape and low surface roughness of the pellets 3 is of particular importance for acid-sensitive active substances in which defects in the isolation caused by satellites or by the excessively rough surface of over-large particles of tartaric acid powder may lead to significantly impaired storage stability and hence durability of the finished product.

The pellets 5 containing active substance are prepared by spraying an active substance suspension 4 onto the isolated tartaric acid cores 3 obtained by the method described hereinbefore. The preparation of the active substance suspension 4 is of particular importance according to the invention. The active substance suspension 4 is prepared using dabigatran etexilate methanesulphonate in the form of its polymorph I. Polymorph I is characterised inter alia by a melting point of Tₘₚ=180±3° C. (determined by DSC; evaluated using peak maximum; heating rate: 10° C./min) The targeted production of polymorph I is possible for example using the method described in WO 05/028468 (cf. particularly Example 1). Where the term active substance is used within the scope of the present invention, unless stated otherwise, this is to be understood as being a reference to polymorph I of dabigatran etexilate methanesulphonate.

In order to prepare the active substance suspension 4 isopropanol is taken and combined with hydroxypropylcellulose with stiffing. The stirring is carried out using a conventional stirrer, for example a propeller stirrer. The stirrer speed is usually in the range from 100-1000 revolutions per minute (rpm), preferably 200-800 rpm, more preferably 300-700 rpm, particularly preferably 400-600 rpm. Isopropanol is preferably used in virtually anhydrous form (99.5%). It is stirred until the hydroxypropylcellulose is completely dissolved. Once the solution is clear, the active substance is added and stirring is continued for 10-60 minutes, preferably for 20-30 minutes. Then talc is added at a constant stiffing rate. Stirring is then carried out again for 10-60 minutes, preferably 10-15 minutes.

Any clumps formed are broken up by homogenisation using a suitable disperser. According to the invention dispersers known in the art having rotation speeds of from 8000 up to 20000 rpm are preferably used for this. This homogenisation is carried out over a period of 0.5-5 hours, preferably 0.5 to 4 hours, particularly preferably 1-3 hours. The homogeneity or freedom from clumps of the suspension 4 is checked regularly, preferably every hour.

To prepare the suspension 4, 0.05 to 0.5 kg, preferably 0.1 to 0.3 kg, particularly preferably 0.15-0.25 kg active substance are used per kilogram of isopropanol put in. The amount of hydroxypropylcellulose used is 0.01 to 0.1 kg, preferably 0.02 to 0.07 kg particularly preferably 0.03-0.05 kg, per kilogram of isopropanol put in. The amount of talc used is 0.005 to 0.07 kg, preferably 0.01 to 0.05 kg, particularly preferably 0.02-0.04 kg, per kilogram of isopropanol put in.

The ratio of active substance to hydroxypropylcellulose is preferably in the range from 3:1 to 7:1, preferably 4:1 to 6:1, particularly preferably about 5:1, with regard to the mass of the two constituents in the active substance suspension according to the invention. The ratio of active substance to talc is preferably in the range from 4:1 to 8:1, preferably 5:1 to 7:1, particularly preferably 6:1 to 6.5:1 with regard to the mass of the two constituents in the active substance suspension according to the invention.

The concentration of the active substance is preferably at 10-25% (w/w), preferably at 11-20% (w/w), particularly preferably at 12-19% (w/w) in the active substance suspension according to the invention. The total concentration of the constituents active substance, hydroxypropylcellulose and talc in the active substance suspension according to the invention is preferably 14-40% (w/w), preferably 15-30% (w/w), particularly preferably 16-25% (w/w).

Within the scope of the present invention, unless stated otherwise, concentrations are always given as percent by weight or mass percent.

Surprisingly it has been found that the temperature selected for the preparation of the suspension 4 has a decisive effect on the characteristics of the final product. In order to guarantee that the manufacturing process reproducibly leads to a product with a defined polymorphic form of the active substance it turned out that the temperature should best be kept below 30° C. throughout the entire manufacturing process. If the suspension 4 is produced or even stored at too high a temperature, this may lead to a change in the polymorphic form of the active substance. Particularly preferably the temperature of the manufacturing process is in the range from 0-30° C., particularly preferably in the range from 5-30° C.

The active substance suspension 4 is stirred further until further processing is carried out, so that no sedimentation occurs. If the suspension is stored at below 30° C., further processing is preferably carried out in the course of not more than 48 h. If the suspension is prepared and stored at 22° C., for example, it is preferably further processed within 60 hours.

In one aspect the present invention relates to a process for preparing a suspension 4 of the polymorph I of dabigatran etexilate methanesulphonate in isopropanol, which is characterised in that the temperature during the manufacture and storage of the suspension is always below 30° C., preferably in the range from 0-30° C., particularly preferably in the range from 5-30° C.

In another aspect the present invention relates to the suspension 4 of the polymorph I of dabigatran etexilate methanesulphonate in isopropanol, which may be obtained by the manufacturing process mentioned above.

In another aspect the present invention relates to the use of the suspension 4 of the polymorph I of dabigatran etexilate methanesulphonate in isopropanol as starting material for preparing a medicament formulation of dabigatran etexilate methanesulphonate.

In another aspect the present invention relates to the use of the active substance suspension 4 according to the invention as starting material for preparing a medicament formulation of dabigatran etexilate methanesulphonate, the suspension 4 having been reacted within 48 h at a storage temperature of less than 30° C.

In another aspect the present invention relates to the use of the active substance suspension 4 according to the invention as starting material for preparing a medicament formulation of dabigatran etexilate methanesulphonate, the suspension 4 having been reacted within 60 h at a storage temperature of less than 22° C.

To prepare the final active substance formulation 5 the active substance suspension 4 obtained by the above process is sprayed onto the isolated tartaric acid cores 3 described hereinbefore.

In another aspect the present invention relates to a process for preparing a medicament formulation of dabigatran etexilate methanesulphonate 5, characterised in that the active substance suspension 4 according to the invention is sprayed onto isolated tartaric acid cores 3.

In another aspect the present invention relates to a medicament formulation of dabigatran etexilate methanesulphonate 5, obtainable by spraying the active substance suspension 4 according to the invention onto isolated tartaric acid cores 3.

To prepare the active substance pellets 5 the isolated tartaric acid pellets 3 are placed in a suitable pan. The pan is preferably a horizontal pan in which the particles are mixed and moved about by the rotation of the pan. Various designs of pan are known in the art. On this subject reference is made for example to the disclosures of EP 80199, WO 83/03052, WO 95/19713 or WO 06/134133.

Preferably, according to the invention, a coater with an unperforated pan is used. In contrast to fluidised bed processes, the suspension is sprayed onto the fluid pellet bed in a rotating pan using the “top spray” method. It is particularly preferred according to the invention for the dry air to be passed into the pellet bed using so-called immersion blades, as described for example in WO2006/134133 (cf. FIGS. 3a and 3b), and discharged through an opening in the back wall of the coater.

In order to achieve good results in terms of homogeneity and uniformity of the active substance pellets 5 obtained, the product temperature, spray pressure, spray rate and amount of supply air in particular should be kept within specified ranges. Monitoring these parameters according to the present invention also ensures limited decomposition of the active substance, a reproducible content of active substance in the pellets 5, associated reduced spray losses and also reduced formation of multiples (clumps of several pellets). A reduced formation of multiples directly influences the yield as clumps would be separated off during the final screening of the active substance pellets 5.

By the product temperature is meant the temperature that prevails in the p, die in the pellet bed. The horizontal pan is first of all charged with the isolated tartaric acid pellets 3 described hereinbefore and the isolated tartaric acid pellets 3 are heated. They are preferably heated to a temperature of 30-50° C., preferably 35-46° C., particularly preferably 40-45° C. Once this temperature has been reached, the active substance suspension 4 described hereinbefore is sprayed on. The horizontal pan usually keeps the pellet bed from 3 in motion at a rate of 3-12 rpm, preferably 4-10 rpm, particularly preferably 6-8 rpm.

By the spray pressure is meant the pressure of compressed air which is used for atomisation at the nozzle through which the active substance suspension 4 is sprayed on. The spray pressure is not dependant on the batch size in the process according to the invention and according to the invention is preferably in the range from 0.5-1.5 bar, preferably 0.7-1.0 bar, particularly preferably 0.8-1.0 bar.

By the spray rate is meant the amount of active substance suspension 4 that is sprayed onto the fluid pellet bed per hour. The spray rate is dependant on the batch size in the process according to the invention. The standardised spray rate according to the invention per kilogram of isolated tartaric acid pellets 3 supplied is preferably in the range from 0.05-0.15 (kg/h)/kg, preferably 0.06-0.09 (kg/h)/kg, particularly preferably 0.062-0.081 (kg/h)/kg.

If for example 320 kg tartaric acid pellets 3 are placed in a batch, a standardised spray rate of 0.062 (kg/h)/kg corresponds to an actual spray rate of 20 kg/h. If for example 32 kg tartaric acid cores 3 are placed in a batch, a standardised spray rate of 0.062 (kg/h)/kg corresponds to an actual spray rate of 2 kg/h.

By the amount of supply air is meant the amount of dry air that is introduced into the fluid pellet bed per hour. The amount of supply air is dependant on the batch size in the process according to the invention. The standardised amount of supply air per kilogram of isolated tartaric acid pellets 3 supplied according to the invention is preferably in the range from 4.5-8.0 (m³/h)/kg. Preferably 5.0-7.3 (m³/h)/kg, particularly preferably 5.5-6.3 (m³/h)/kg.

If for example 320 kg tartaric acid pellets 3 are placed in one batch, a standardised amount of supply air of 5.5 (m³/h)/kg corresponds to an actual amount of supply air of 1760 m³/h. If for example 32 kg tartaric acid cores are placed in one batch, a standardised amount of supply air of 7.2 (m³/h)/kg corresponds to an actual amount of supply air of 1760 m³/h.

The temperature of the supply air fed in according to the invention is preferably below 90° C., particularly preferably below 80° C. Ideally the temperature of the supply air should be in the range from 40° -75° C.

Once the spray process has ended the subsequent drying of the active substance pellets 5 takes place in the horizontal pan rotating at a rate of 1-10 rpm, preferably 2-8 rpm, particularly preferably 4-6 rpm, at a supply air temperature of at least 20° C., preferably at least 25° C., particularly preferably in the range from 30-50° C. The standardised amount of supply air during the drying process per kilogram of tartaric acid pellets 3 originally isolated according to the invention is preferably in the range from 1.0-4.0 (m³/h)/kg, preferably 1.2-3.5 (m³/h)/kg, particularly preferably 1.5-3.2 (m³/h)/kg. The drying time in the horizontal pan according to the invention is preferably in the range from 30 minutes to 5 hours, preferably 45 minutes to 4 hours. Especially in industrial production units and batch sizes over 100 kg (based on the isolated tartaric acid pellets 3 used) the drying time is particularly preferably in the range from 1-2 hours.

The amount of active substance suspension 4 sprayed on under the prevailing conditions depends not only on the active substance concentration in the suspension 4 but also on the batch size of the isolated tartaric acid pellets 3 supplied and the desired quantity of active substance per final active substance pellet (so-called charge). Particularly preferably the active substance charge per active substance pellet 5 is in the range from 15-50% (w/w). Particularly preferred active substance pellets 5 according to the invention have a charge of active substance of 20-45% (w/w), particularly preferably 36-42% (w/w).

With smaller batches, the isolated tartaric acid pellets 3 can be charged with virtually any desired amount of active substance in a single process step. If a particularly preferred active substance suspension 4 with an active substance concentration of about 15% (w/w) and an overall concentration of the constituents active substance, hydroxypropylcellulose and talc of about 20% (w/w) is used according to the invention, a desired charge of for example 24% of active substance per active substance pellet 5 with a supply of 1 kg of isolated tartaric acid pellets 3 requires the use of about 2.45 kg of active substance suspension 4 according to the invention. It may possibly make sense to use active substance suspension 4 in an excess of up to 15%, to compensate for any spray losses that may occur. In such cases it may be advisable to set the amount at 2.81 kg 5 instead of the above-mentioned 2.45 kg 5, for the desired charge of 24%. If a charge of 40% (w/w) is desired when using the same suspension, about 6.03 kg of suspension 4 have to be sprayed onto 1 kg of isolated tartaric acid pellets 3. It may also possibly be advisable here to use the active substance suspension 4 in an excess of up to 15%, to compensate for any spray losses that may occur.

In the event of a greater charge of the isolated tartaric acid pellets 5 the total weight of the batch and, in the present case, in particular the volume naturally increases constantly during the spraying of the active substance suspension 4. A charge of for example 40% of the isolated tartaric acid pellets 3 with active substance leads to roughly a doubling of the total weight and an increase in bulk density by a factor of approx. 1.3 (i.e. an even greater increase in volume in relation to the mass) of the material for spraying 5. This sharp increase in the mass and particularly the volume of the material for spraying 5 may negatively affect the spray process in large industrial batches, as for example uniform drying of the spray material 5 can no longer be achieved easily or without complex technical procedures.

For high charges it may therefore be helpful, for large batch sizes, to carry out the spray process in a number of stages, each of which results in different charging levels with a comparable amount of material supplied for spraying. Preferably, according to the invention, the process is carried out in up to 5, preferably up to 4, particularly preferably up to 3 stages. A proportion of the material for spraying obtained at the end of each respective stage is fed into the next spray process in each case. Sufficient spray material containing active substance is taken from the previously obtained spray material and introduced into the next step of the process to ensure that the mass of spray material supplied at the beginning of the respective spray process is always roughly the same. The charging with active substance is increased from step to step.

Particularly preferably, according to the invention, the same active substance suspension is used for all the spray processes.

Particularly preferably, according to the invention, a two-stage process is carried out. In a first process step, pellets 5 are prepared containing an active substance with a charge level of about 10-35% (w/w), preferably about 15-30% (w/w), particularly preferably about 20-25% (w/w). Then, of the batch of active substance pellets 5 thus obtained, 50-80%(w/w), preferably about 55-75% (w/w), particularly preferably about 65-70% (w/w) are separated off and fed into a new spray process as material for spraying. These pellets 5 already containing active substance are then sprayed with the active substance suspension 4 under the above-mentioned spray conditions in a new spray process. Particularly preferred active substance pellets 5 according to the invention have a charge of active substance of 35-45% (w/w), particularly preferably 38-42% (w/w), after this second spray process.

In another aspect the present invention relates to a medicament formulation of dabigatran etexilate methanesulphonate 5, obtainable by spraying the active substance suspension 4 according to the invention onto isolated tartaric acid cores 3 by the method described hereinbefore.

To eliminate and clumps that may have formed, the active substance pellets thus obtained are screened through screens of a defined mesh size. The mesh size selected naturally depends on the charging of the respective active substance pellets. For lower charges, closer-meshed screens may be used. On this subject, reference is made to the explanations by way of example in the experimental section that follows.

Finally, the active substance pellets obtained are packed into commercially obtainable capsules, preferably into commercially obtainable HPMC capsules.

The Examples that follow serve to illustrate the present invention in more detail.

Determining the Particle Sizes of tartaric Acid by Air Let Screening

Measuring Device and Settings:

Measuring device: Air jet screen, e.g. Alpine A 200 LS

Screens: As required

Weight put in: 10 g/screen

Duration: 1 min/screen, then 1 min each up to the maximum weight loss of 0.1 g

Preparation of Sample/Supply of Product:

The substance is transferred into a mortar and any lumps present are destroyed by intensive pounding. The screen with rubber seal and cover is placed on a balance, set to zero and 10.0 g of the pounded substance are weighed onto the screen.

The screen together with its contents, rubber seal and cover are placed on the device. The timer is set to 1 minute and the material is treated by air jet screening for this time. Then the residue is weighed out and documented. This process is repeated until the decrease in the weight of the residue after air jet screening is <0.1 g.

### EXAMPLE 1

### Preparation of the Starter Pellets

480 kg water are heated to 50° C. and 120 kg of acacia (gum arabic) are added with stirring in a conventional mixing container having a dished end and stirrer. Stirring is continued at constant temperature until a clear solution is obtained. Once there is a clear solution (usually after 1 to 2 hours) 600 kg tartaric acid are added with stirring. The tartaric acid is added at constant temperature while stirring is continued. After the addition has ended the mixture is stirred for about another 5 to 6 hours.

1000 kg tartaric acid are added to a slowly rotating (3 revolutions per minute) unperforated horizontal pan with a spraying and powder applying unit (e.g. Driamat 2000/2.5). Before spraying starts, a sample of the acid is taken for screening analysis. The acid in question is tartaric acid particles with a particle size in the range from 0.4-0.6 mm

The acid rubber solution obtained by the above method is sprayed onto the tartaric acid particles thus provided. During the spraying, the quantity of air supplied is adjusted to 1000 m³/h and 35°-75° C. The differential pressure is 2 mbar and the speed of rotation of the pan is 9 revolutions per minute. The nozzles should be arranged at a distance of 350-450 mm from the filling.

The acid rubber solution is sprayed on by alternating with the following steps. After about 4.8 kg of the acid rubber solution has been sprayed onto the tartaric acid particles of particle size 0.4-0.6 mm and the solution has been distributed, about 3.2 kg tartaric acid powder are sprinkled onto the damp tartaric acid particles. The tartaric acid powder in question consists of fine tartaric acid particles with a particle size of <50 microns. In all, 800 kg tartaric acid powder are required. After the said tartaric acid powder has been sprinkled on and distributed the spray material is dried until a product temperature of about 40° C. is reached. This is in turn followed by the spraying on of the acid rubber solution.

These cycles are repeated until the acid rubber solution is used up. Once the process has ended the acid pellets are dried in the pan at 3 rpm for 240 minutes. To prevent caking after the drying has finished, an intermittent program is run at 3 rpm for 3 minutes every hour. In the present instance this means that the pan is rotated at 3 rpm for 3 minutes at intervals of one hour and then left to stand. The acid pellets are then transferred into a dryer. They are then dried at 60° C. over a period of 48 hours. Finally, the particle size distribution is determined by screen analysis. The particle size with a diameter of 0.6-0.8 mm corresponds to the product. This fraction should make up >85%.

### EXAMPLE 2

### Isolation of the Starter Pellets

To prepare the isolating suspension, 666.1 (347.5) kg of ethanol are placed in the mixing container and the hydroxypropylmethylcellulose (33.1 (17.3) kg) is added with stirring at approx. 600 rpm and dissolved. Then under the same conditions 0.6 (0.3) kg dimeticone are added. Shortly before use, talc (33.1 (17.3) kg) is added, again with stirring, and suspended.

The acid pellets 1200 (600) kg are poured into the coating apparatus (e.g. GS-Coater Mod. 600/Mod. 1200) and sprayed therein in the rotating pan with the isolating suspension described above in a continuous spraying process lasting several hours at a spraying rate of 32 kg/h for the 1200 kg mixture or 21 kg/h for the 600 kg mixture. The pellets are also dried continuously with an air supply at up to 70° C.

After the GS-Coater has been emptied, the isolated starter pellets are fractionated by screening. The product fraction with a diameter ≦1.0 mm is stored and used further.

### EXAMPLE 3

### Preparation of the Dabigatran Etexilate Suspension

26.5 kg hydroxypropylcellulose are added to 720 kg isopropanol in a 1200 litre mixing container fitted with a propeller stirrer and the mixture is stirred until fully dissolved (about 12-60 hours; roughly 500 rpm). Once the solution is clear, 132.3 kg of dabigatran etexilate methanesulphonate (polymorph I) are added with stirring (400 rpm) and the mixture is stirred for about another 20-30 minutes. Then 21.15 kg of talc is added at a constant stirring rate and stirring is continued at the same speed for about another 10-15 minutes. The steps described above are preferably carried out under a nitrogen atmosphere.

Any clumps formed are broken up by homogenising using an UltraTurrax stirrer (about 60-200 minutes). The suspension temperature should not exceed 30° C. throughout the entire manufacturing process.

The suspension is stirred until ready for further processing to ensure that no sedimentation occurs (at roughly 400 rpm).

If the suspension is stored at below 30° C., it should be further processed within at most 48 h. If for example the suspension is manufactured and stored at 22° C., it may be further processed within 60 hours. If the suspension is stored for example at 35° C. it should be further processed within at most 24 h.

### EXAMPLE 4

### Preparation of the Dabigatran Etexilate Active Substance Pellets

A horizontal pan with an unperforated container is used (GS Coater Mod. 600). In contrast to the fluidised bed method, the suspension is sprayed onto the fluidised bed of pellets in the rotating pan by the “top spray” method. It is sprayed on through nozzles 1.4 mm in diameter. The dry air is passed into the bed of pellets through so-called immersion blades and transported away through an opening in the back wall of the coater.

The horizontal pan is charged with 320 kg of the tartaric acid pellets obtained according to Example 2 and the bed of pellets is heated up. Once a product temperature of 43° C. has been reached, spraying begins. 900 kg of the suspension prepared previously according to Example 3 are sprayed on, first of all for 2 h at a spraying rate of 20 kg/h, then 24 kg/h and a spray pressure of 0.8 bar. The suspension is stirred constantly. The temperature of the air supplied is at most 75° C. The amount of air supplied is about 1900 m³/h.

Then the pellets are dried in the horizontal pan (5 revolutions per minute) at an air inflow temperature of at least 30° C., at most 50° C. and an air inflow amount of 500 m³/h over a period of about 1-2 hours.

325 kg of the pellets thus obtained are then loaded once more into a horizontal pan and heated to 43° C. 900 kg of the suspension prepared previously according to Example 3 are sprayed on, first of all for 2 h at a spraying rate of 20 kg/h, then 24 kg/h and a spray pressure of 0.8 bar. The suspension is stirred constantly. The temperature of the air supplied is at most 75° C. The amount of air supplied is about 1900 m³/h.

Then the pellets are dried in the horizontal pan (5 revolutions per minute) at an air inflow temperature of at least 30° C., at most 50° C. and an air inflow amount of 500 m³/h over a period of about 1-2 hours.

The dried pellets are then passed through a vibrating screen with a mesh size of 1 6 mm and stored in containers with desiccants until needed for further processing.

### EXAMPLE 5

### Examples of Formulations

The following examples of formulations are then obtained from the active substance pellets obtained according to Example 4 by packing into hydroxypropylmethylcellulose capsules:

| | | amount [mg] |
|---|---|---|
| Ingredient | amount [mg] per capsule | per capsule |
| active substance I | 86.48⁽¹⁾ | 126.83⁽²⁾ |
| Acacia (gum arabic) | 4.43 | 6.50 |
| tartaric acid | 88.56 | 129.9 |
| hydroxymethyl- | 2.23 | 3.27 |
| propylcellulose 2910 | | |
| dimethylpolysiloxane 350 | 0.04 | 0.06 |
| talc | 17.16 | 25.16 |
| hydroxypropylcellulose | 17.30 | 25.37 |
| HPMC capsule | 60⁽³⁾ | 70⁽⁴⁾ |
| Total | 276.2 | 387.1 |

⁽¹⁾corresponds to 75 mg of free active substance base
⁽²⁾corresponds to 110 mg of free active substance base
⁽³⁾weight of capsule size is about 60 mg
⁽⁴⁾weight of capsule size is about 70 mg

In another aspect the present invention relates to one of the above-mentioned medicament formulations as such.

In another aspect the present invention relates to a medicament formulation which contains 60-90 mg, preferably 70-80 mg, particularly preferably about 75 mg of dabigatran etexilate of formula I. In another aspect the present invention relates to a medicament formulation which contains 90-130 mg, preferably 100-120 mg, preferably 105-115 mg, particularly preferably about 110 mg of dabigatran etexilate of formula I.

In another aspect the present invention relates to a medicament formulation which contains 60-90 mg, preferably 70-80 mg, particularly preferably about 75 mg of dabigatran etexilate of formula I in the form of the polymorph I of its methanesulphonate. In another aspect the present invention relates to a medicament formulation which contains 90-130 mg, preferably 100-120 mg, preferably 105-115 mg, particularly preferably about 110 mg of dabigatran etexilate of formula I in the form of the polymorph I of its methanesulphonate.

In another aspect the present invention relates to a medicament formulation which also contains hydroxymethylpropylcellulose, besides dabigatran etexilate of formula I in the form of the polymorph I of its methanesulphonate.

In another aspect the present invention relates to a medicament formulation which also contains dimethylpolysiloxane besides dabigatran etexilate of formula I in the form of the polymorph I of its methanesulphonate.

In another aspect the present invention relates to a medicament formulation which also contains the constituents gum arabic, tartaric acid, hydroxymethylpropylcellulose, dimethylpolysiloxane, talc as well as hydropropylcellulose, besides dabigatran etexilate of formula I in the form of the polymorph I of its methanesulphonate.

In another aspect the present invention relates to a medicament formulation which contains exclusively the constituents gum arabic, tartaric acid, hydroxymethylpropylcellulose, dimethylpolysiloxane and talc as well as hydropropylcellulose, besides dabigatran etexilate of formula I in the form of the polymorph I of its methanesulphonate.

In another aspect the present invention relates to a medicament formulation which contains 60-90 mg, preferably 70-80 mg, particularly preferably about 75 mg of dabigatran etexilate of formula I, for the post-operative prevention of deep vein thromboses and in stroke prevention, particularly for preventing strokes in patients with atrial fibrillation. In another aspect the present invention relates to a medicament formulation which contains 90-130 mg, preferably 100-120 mg, preferably 105-115 mg, particularly preferably about 110 mg of dabigatran etexilate of formula I, for the post-operative prevention of deep vein thromboses and in stroke prevention, particularly for preventing strokes in patients with atrial fibrillation.

1) A process for preparing a suspension 4 of polymorph I of methanesulphonic acid salt of dabigatran etexilate of formula I (active substance) 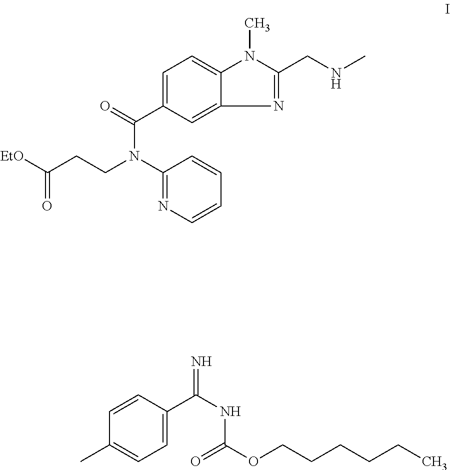
comprising the step of preparing a suspension of polymorph I of dabigatran etexilate methanesulfonate with talc in a solution of hydroxypropylcellulose in isopropanol, wherein the suspension is prepared at a temperature not exceeding 30° C.

2) The process according to claim 1, the hydroxypropylcellulose is first dissolved in isopropanol and the polymorph I of dabigatran etexilate methanesulphonate and talc are then suspended in the hydroxypropylcellulose solution.

3) The process according to claim 1, wherein 0.05 to 0.5 kg dabigatran etexilate methanesulphonate is used per kilogram of isopropanol used.

4) The process according to one of claim 1, wherein 0.01 to 0.1 kg hydroxypropylcellulose is used per kilogram of isopropanol used.

5) The process according to one of claim 1, wherein 0.005 to 0.07 kg talc is used per kilogram of isopropanol used.

6) A suspension 4, made by the process according to claim 1.

7) A suspension 4 according to claim 6, wherein the concentration of active substance is 10-25% (w/w).

8) The suspension 4 according to claim 6, wherein the overall concentration of the active substance, hydroxypropylcellulose and talc is 14-40% (w/w).

9) The use of a suspension 4 according to claim 6 for use as a starting material for preparing dabigatran etexilate methanesulphonate pellets 5.

10) A process for preparing dabigatran etexilate methanesulphonate pellets 5, comprising the step of spraying the suspension 4 according to claim 1 onto isolated tartaric acid cores to form isolated tartaric acid pellets 3.

11) The process for preparing dabigatran etexilate methanesulphonate pellets 5 according to claim 10, wherein a temperature of the tartaric acid pellets 3 supplied is adjusted to 30-50° C.

12) The process for preparing dabigatran etexilate methanesulphonate pellets 5 according to claim 10, wherein the suspension 4 is sprayed onto the tartaric acid pellets 3 at a standardized spray rate in the range of 0.05-0.15 (kg/h) per kilogram of tartaric acid pellets 3 used.

13) The process for preparing dabigatran etexilate methanesulphonate pellets 5 according to claim 10, further compromising dry air into pellet bed in the range from 4.5-8.0 (m³/h per kilogram of tartaric acid pellets 3 used.

14) The process for preparing dabigatran etexilate methanesulphonate pellets 5 according to claim 10, wherein the tartaric acid pellets 3 are obtained by spraying an isolating suspension 2 onto tartaric acid cores 1, wherein 2 is an ethanolic isolating suspension comprising hydroxypropylmethylcellulose.

15) The process for preparing dabigatran etexilate methanesulphonate pellets 5 according to claim 14, wherein the ethanolic isolating suspension 2 further comprises talc.

16) The process for preparing dabigatran etexilate methanesulphonate pellets 5 according to claim 15, wherein the ethanolic isolating suspension 2 further comprises dimethylpolysiloxane.

17) Dabigatran etexilate methanesulphonate pellets 5 made by the process according to claim 10.
